(19) United States
(12) Patent Application Publication
(10) Pub. No.: US 2022/0218755 A1
(43) Pub. Date: Jul. 14, 2022

(54) EXTRACELLULAR VESICLES AND THEIR USES

(71) Applicant: United Therapeutics Corporation, Silver Spring, MD (US)

(72) Inventors: Roger ILAGAN, Durham, NC (US); Jordan SHIN, Lexington, MA (US); Donald BRYANT, Wilson, NC (US); Elizabeth Moore EADES, Efland, NC (US)

(73) Assignee: United Therapeutics Corporation, Silver Spring, MD (US)

(21) Appl. No.: 17/572,164

(22) Filed: Jan. 10, 2022

Related U.S. Application Data
(60) Provisional application No. 63/136,179, filed on Jan. 11, 2021.

(51) Int. Cl. A61K 35/28; A61P 37/00; A61K 9/51; A61P 9/00

(52) U.S. Cl. CPC A61K 35/28; A61P 9/00; A61K 9/5176; A61P 37/00

(57) ABSTRACT

Provided are methods for treating or preventing myocarditis by administering extracellular vesicles, including exosomes. In some embodiments, the EVs are from bone marrow-derived mesenchymal stem (or stromal) cells. The myocarditis can be the result of viral infections such as Coxsackievirus B virus, SARS-CoV-2, or other viruses.

### CROSS-REFERENCE TO RELATED APPLICATIONS

This application claims priority under 35 U.S.C. § 119 to U.S. provisional application 63/136,179 filed Jan. 11, 2021, the entire contents of which are incorporated herein by reference.

### FIELD OF USE

The present application relates treating or preventing myocarditis by administering extracellular vesicles, including exosomes. In some embodiments, the EVs are from bone marrow-derived mesenchymal stem (or stromal) cells. The myocarditis can be the result of viral infections such as Coxsackievirus B virus, SARS-CoV-2, or other viruses.

### BACKGROUND

Myocarditis is a condition characterized by inflammation of the heart, and patients with this condition may present with a range of symptoms including shortness of breath, chest pain, distinct ECG changes, acute heart failure, and dilated cardiomyopathy (DCM; myocarditis may be present in as many as 9-50% of DCM cases). Myocarditis can stem from a wide spectrum of causes including reactions to toxic drugs, autoimmunity, and infection (e.g., viral, bacterial, fungal) with the latter being the most frequent cause of the condition. In Western countries specifically, viral infection (e.g., enteroviruses such as Coxsackievirus B virus, SARS-CoV-2) is the most common cause of myocarditis and one of the most well-studied forms of the condition.

In general, viral myocarditis is characterized by three stages: 1) viral entry and replication (acute stage), 2) inflammatory cell infiltration (subacute stage), and 3) cardiac remodeling (chronic stage). The extent of injury caused by viral infection as well as damage from the host's inflammatory reaction are often considered to be the key drivers of the pathogenesis of viral myocarditis. However, studies have identified other mechanisms such as damage to the cardiac vasculature, dysfunctional cellular metabolism, and aberrant cellular salvage (e.g., apoptosis) as playing important roles in the pathogenesis of this condition.

Given the multiple pathogenic processes that can drive the progression of myocarditis, there is a significant need for therapies with mechanisms of action that can address multiple facets of this condition.

### SUMMARY OF THE INVENTION

The present disclosure is directed to a method of treating, preventing, or reducing the severity of myocarditis, comprising administering to a subject in need thereof an effective dose of an isolated extracellular vesicle (EV). In some embodiments, the myocarditis is caused by an infection, a toxic drug, or an autoimmune disease or disorder. In some embodiments, the infection is a bacterial infection, a fungal infection, or a viral infection. In some embodiments, the viral infection is caused by Coxsackievirus B virus, or SARS-CoV-2. In some embodiments, the viral infection is caused by Adenoviruses, influenza, parvoviruses and herpes viruses. In some embodiments, the method treats, precents, or reduced the severity of myocarditis caused by SARS-CoV-2 infection.

In one aspect, the method treats, prevents or reduces the severity of the heart condition or disorder by reducing inflammation in the heart.

In another embodiment, there is an isolated EV comprising one or more proteins that treats, prevents or reduces the severity of the heart condition or disorder by reducing inflammation in the heart.

### BRIEF DESCRIPTION OF THE DRAWINGS

The provided drawings exemplify, but do not limit, the disclosed subject matter.

FIG. 1 shows that UNEX-42 EVs inhibit lipopolysaccharide (LPS)-Induced TNFα (left panel) and Chemokine (C-X-C Motif) Ligand 1 (GRO; right panel) in THP-1 monocytes. LPS is abbreviation for lipopolysaccharide; TNFα is abbreviation for tumor necrosis factor alpha. PBS is abbreviation for phosphate buffered saline.

FIGS. 2A and 2B show that UNEX-42 EVs increase expression of expression of CD206 (left panel) and IL10 (right panel) in M2-polarized macrophages.

FIGS. 3A-3D show that UNEX-42 EVs inhibit LPS-induced expression of neutrophil chemoattractant KC (FIG. 3A) and LIX (FIG. 3B) in a mouse lung injury model. FIG. 3C shows that UNEX-42 EVs increases arterial blood oxygen reduced by LPS. FIG. 3D shows that UNEX-42 EVs improves LPS-induced lung injury.

FIG. 4 shows secretion of pro-inflammatory factors in the bronchoalveolar lavage (BAL) in a rat model of acute lung injury induced by intratracheal administration of LPS. UNEX-42 decreases the levels of pro-inflammatory factors in a dose-dependent manner.

FIG. 5 shows that UNEX-42 EVs inhibit LPS-induced macrophages, lymphocytes and neutrophils infiltration in the bronchoalveolar lavage (BAL).

FIG. 6 shows that UNEX-42 EVs inhibit bleomycin or silica induced macrophages, lymphocytes and neutrophils infiltration in the bronchoalveolar lavage (BAL).

FIG. 7 shows that UNEX-42 EVs increased the presence of CD8-positive cytotoxic T cells in a mouse model of H1N1 influenza-induced lung injury.

FIG. 8 shows that UNEX-42 EVs promote endothelial cell vessel formation (left panel) and stabilization as determined by number of branching points (right panel).

FIGS. 9A-9B show that UNEX-42 EVs increase oxygen consumption (FIG. 9A) and glucose uptake (FIG. 9B), and decrease lactate accumulation (FIG. 9B) in smooth muscle cells cultured under low oxygen conditions. OCR is abbreviation for oxygen consumption rate.

FIG. 10 shows that UNEX-42 EVs inhibit hyperoxia-induced cell stress and apoptosis as measured by cytochrome c release in an alveolar epithelial cell line (left panel), cellular content (middle panel), and TNFα release (right panel).

### DETAILED DESCRIPTION

The present disclosure meets the need of providing improved methods of treatment of myocarditis. EV-based therapy is one class of treatments that may have significant therapeutic potential for myocarditis based on their ability to exert beneficial effects via multiple mechanisms.

UNEX-42 is a preparation of extracellular vesicles (EVs) that are secreted from primary non-immortalized human bone marrow-derived mesenchymal stem (or stromal) cells (BM MSCs). The UNEX-42 EVs and their preparation and isolation are described in detail in U.S. provisional application 62/943,555 and the U.S. non-provisional application Ser. No. 17/109,775, which are incorporated herein in their entirety.

EVs, including UNEX-42 EVs, can be used in methods of treating, preventing, or reducing the severity of myocarditis. In particular, present disclosure relates to use of EVs, including UNEX-42 EVs, for immune modulation, promotion of angiogenesis, improvement in cellular metabolism, and improvement in cellular salvage (apoptosis) to treat, prevent, or reduce the severity of myocarditis. In some embodiments, the myocarditis is caused by an infection, a toxic drug, or an autoimmune disease or disorder. In some embodiments, the infection is a bacterial infection, a fungal infection, or a viral infection. In some embodiments, the viral infection is caused by Coxsackievirus B virus, or SARS-CoV-2. In some embodiments, the viral infection is caused by Adenoviruses, influenza, parvoviruses and herpes viruses In some embodiments, the method treats myocarditis caused by SARS-CoV-2 infection.

Inflammation

An inflammatory cytokine is a type of cytokine that is secreted from immune cells and certain other cell types that promote inflammation. Inflammation may be caused by cellular stress such as oxidative stress, toxins, or infections.

Inflammatory cytokines are predominantly produced by T helper cells (Th) and macrophages and involved in the upregulation of inflammatory reactions. Therapies to treat inflammatory diseases include monoclonal antibodies that either neutralize inflammatory cytokines or their receptors.

Inflammatory cytokines or chemokines may include interleukin-1 (IL-1), IL-3, IL-6 and IL-18, tumor necrosis factor alpha (TNF-α), interferon gamma (IFNγ), and granulocyte-macrophage colony stimulating factor (GM-CSF), Chemokine (C-X-C Motif) Ligand 1 (GRO), Chemokine (C-C Motif) Ligand 21 (6Ckine), Granulocyte Chemotactic Protein 2 (GCP2), or Chemokine (C-X-C Motif) Ligand 16 (CXCL16), macrophage inflammatory protein 1a (MIP1a), macrophage inflammatory protein 1b (MIP1b), interleukin 1 beta (IL1(3), interleukin 12 beta (IL12(3), or interferon-inducible T-cell alpha chemoattractant (ITAC). This inflammatory state of the lung is attributed to barotrauma associated with mechanical ventilation, and oxidative stress that results from high oxygen supplementation. Accordingly, the immunomodulatory activity of EVs derived from MSC may be evaluated by measuring levels of pro-inflammatory cytokines such as IL-3 or tumor necrosis factor alpha (TNF-α).

In some embodiments, the EVs of the present disclosure may prevent secretion of pro-inflammatory cytokines. In some embodiments, the pro-inflammatory cytokines comprise IL-3 or tumor necrosis factor alpha (TNF-α). In some embodiments, the EVs of the present disclosure may treat acute inflammation. EVs can enhance the presence or activation of anti-inflammatory cytokines, such as mannose receptor (CD206) and interleukin 10 (IL10). The innate immune response is activated following an infection or other insult to the myocardium. In particular, cells of the innate immune system (e.g., macrophages) as well as those of the heart (e.g., cardiomyocytes) are activated by pattern recognition receptors (e.g., Toll-like receptors), which recognize molecular patterns associated with pathogens or damaged cells. Once activated, the innate immune cells as well as cardiomyocyte release a variety of inflammatory factors including cytokines, chemokines, interferons and alarmins. In turn, the secretion of these factors leads to the recruitment of additional innate immune cells (e.g., neutrophils, dendritic cells, monocytes, macrophages) to the inflamed myocardium.

Accordingly, in one aspect of the present disclosure, the method treats, prevents or reduces the severity of the heart condition or disorder by reducing inflammation in the heart. In some embodiments, the EVs reduce inflammation in the heart by reducing a level of a pro-inflammatory factor. In some embodiments, the pro-inflammatory factor comprises TNF-α, IL-1, IL-6, IL-8, TGFβ, IFNγ, C-reactive protein (CRP), RAGE, or combinations thereof. In some embodiments, the EVs reduce inflammation in the heart by increasing a level of an anti-inflammatory factor. In some embodiments, the anti-inflammatory factor comprises IL-10.

In some embodiments, the EVs are capable of decreasing the levels of pro-inflammatory cytokines in a subject by at least about 10%, 15%, 20%, 25%, 30%, 35%, 40%, 45%, or 50% compared to a control left untreated with the EVs. The measured pro-inflammatory cytokines may for example be tumor necrosis factor alpha or interleukin-3.

In some embodiments, the EVs are capable of increasing the levels of anti-inflammatory cytokines in a subject by at least about 10%, 15%, 20%, 25%, 30%, 35%, 40%, 45%, 50%, 60%, 70%, 80%, 90%, 100%, 200% or more compared to a control left untreated with the EVs. The measured pro-inflammatory cytokines may for example be CD206 or interleukin-10 (IL10).

In another aspect, the method reduces infiltration of an immune cell to the heart. In some embodiments, the immune cell comprises neutrophils, dendritic cells, monocytes, macrophages, or lymphocytes.

Vascular Integrity

Viruses (e.g., Coxsackievirus) are capable of infecting the heart's microvascular endothelium, cause damage and permeability of the microvasculature. Myocarditis may exhibit markers of endothelial damage, intramyocardial edema increased vascular permeability, and disruption of the vascular barrier.

Accordingly, in another aspect the present disclosure provide that the method reduces vascular damage in the heart of the subject. In some embodiments, the vascular damage is reduced by endothelial cell vessel formation or stabilization. In some embodiments, the vascular damage is caused by a viral infection. In some embodiments, the viral infection is caused by Coxsackievirus B virus, or SARS-CoV-2.

In some embodiments, the EVs are capable of increasing the total branching points of blood vessels in heart tissue in a subject by at least about 10%, 15%, 20%, 25%, 30%, 35%, 40%, 45%, or 50% compared to a control left untreated with the EVs.

Disrupted Cell Metabolism

Myocarditis disrupts cellular metabolism. For example, pathological conditions such as endotoxic shock can lead to dysregulation of lactate control in heart tissue. In one aspect, the present method improves cellular metabolism in the heart of the subject. In some embodiments, the improved cellular metabolism is measured by oxygen consumption, glucose uptake, or lactate accumulation.

Dysfunctional Cell Salvage

Pathological conditions can trigger apoptosis in cardiomyocytes. For example, high intracellular lactate may drive increased levels of reactive oxygens species which can result in oxidate stress and mitochondrial damage that trigger the mitochondrial-mediated apoptotic pathway, resulting in cytochrome c release. Accordingly, in some embodiments, a method of the present disclosure treats myocarditis by preventing apoptosis. In some embodiments, prevention of apoptosis is determined by measuring cytochrome c release.

In some embodiments, the EVs are capable of decreasing the levels of cytochrome C release in a subject by at least about 10%, 15%, 20%, 25%, 30%, 35%, 40%, 45%, or 50 compared to a control left untreated with the EVs.

EVs

Any suitable EVs can be used, including the UNEX-42 EVs used in the present disclosure are described in detail in U.S. provisional application No. 62/943,555 and the U.S. non-provisional application Ser. No. 17/109,775. Briefly, in some embodiments, the UNEX-42 EVs contain one or more proteins selected from the group consisting of KRT19, TUBB, TUBB2A, TUBB2B, TUBB2C, TUBB3, TUBB4B, TUBB6, CFL1 (HEL-S-15), VIM, EEF1A1, EEF1A1P5, PTI-1, EEF1A1L14, EEFA2, ENPP1, NTSE, HSPA8 (HEL-S-72p), RAB10, CD44, MMP2, CD109, and DKFZp686P132. In some preferred embodiments, the EVs contain one or more proteins selected from the group consisting of CD44, CD109, NTSE, and HSPA8.

EVs can be further assessed for containing the protein markers Syntenin-1, Flotillin-1, CD105, Major histocompatibility complex class I, and members of the tetraspanin family. Accordingly, the EVs further comprise Syntenin-1, Flotillin-1, CD105, and/or Major Histocompatibility complex class I. In some further embodiments, wherein the isolated EV further comprises a member of the tetraspanin family. In some embodiments, the member of the tetraspanin family comprises CD63, CD81, and CD9.

### Definitions

Unless otherwise specified, “a” or “an” means “one or more.”

Unless specifically defined otherwise, all technical and scientific terms used herein shall be taken to have the same meaning as commonly understood by one of ordinary skill in the art.

Unless otherwise indicated, the molecular biology, recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present), and are incorporated herein by reference.

As used herein, the term “subject” (also referred to herein as a “patient”) includes warm-blooded animals, preferably mammals, including humans. In a preferred embodiment, the subject is a primate. In an even more preferred embodiment, the subject is a human.

As used herein the terms “treating”, “treat,” or “treatment” include reducing, mitigating, or eliminating at least one symptom of a disease or condition.

As used herein the terms “preventing”, “prevent” or “prevention” include stopping or hindering the appearance or existence of at least one symptom of a disease or condition, such as vasculopathy. Alternatively, the terms “preventing”, “prevent” or “prevention” may include stopping or hindering the appearance or existence of at least one symptom of a disease or condition, such as dysfunctional angiogenesis, apoptosis, inflammation, mitochondrial dysfunction.

As used here, the term “expression” means RNA expression and/or protein expression level of one or more genes. In other words, the term “expression” can refer to either RNA expression or protein expression or a combination of the two. As used herein, the term contain or containing may include protein and/or RNA expression.

As used here, the term “hypoxia” refers to a condition with an oxygen (02) concentration below atmospheric 02 concentration, 21%. In some embodiments, hypoxia refers to a condition with 02 concentration that is between 0% and 10%, between 0% and 5% 02, between 5% and 10%, or between 5% and 15%. In one embodiment, hypoxia refers to a concentration of oxygen of about 10% 02.

As used here, the term “normoxia” refers to a condition with a normal atmospheric concentration of oxygen, around 20% to 21% 02.

As used here, the terms “isolating” or “isolated,” when used in the context of an extracellular vesicle isolated from a cell culture or media, refers to an extracellular vesicle that, by the hand of man, exists apart from its native environment.

As used here, the term “extracellular vesicles”, abbreviated as EVs, encompass exosomes. The terms “extracellular vesicles” and “EVs,” as used herein, may in some embodiments refer to a membranous particle having a diameter (or largest dimension where the particles is not spheroid) of between about 10 nm to about 5000 nm, more typically between 30 nm and 1000 nm, and most typically between about 50 nm and 750 nm. Most commonly, EVs will have a size (average diameter) that is up to 5% of the size of the donor cell. Therefore, especially contemplated EVs include those that are shed from a cell. Preferred EVs are described in U.S. provisional application No. 62/943,555 and the U.S. non-provisional application Ser. No. 17/109,775.

As used here, the term “population of extracellular vesicles” refers to a population of extracellular vesicles having a distinct characteristic or set of characteristics. The terms “population of extracellular vesicles” and “extracellular vesicles” can be used interchangeably to refer to a population of extracellular vesicles having a distinct characteristic or set of characteristics.

As used here, the term “mesenchymal stromal cell” includes mesenchymal stem cells. Mesenchymal stem cells are cells found in bone marrow, blood, dental pulp cells, adipose tissue, skin, spleen, pancreas, brain, kidney, liver, heart, retina, brain, hair follicles, intestine, lung, lymph node, thymus, bone, ligament, tendon, skeletal muscle, dermis, and periosteum. Mesenchymal stem cells are capable of differentiating into a large number of cell types including, but not limited to, adipose, osseous, cartilaginous, elastic, muscular, and fibrous connective tissues. The specific lineage-commitment and differentiation pathway entered into by mesenchymal stem cells depends upon various influences, including mechanical influences and/or endogenous bioactive factors, such as growth factors, cytokines, and/or local micro environmental conditions established by host tissues. Mesenchymal stem cells are thus non-hematopoietic progenitor cells that divide to yield daughter cells that are either stem cells or are precursor cells which in time will irreversibly differentiate to yield a phenotypic cell.

Treatment Using Extracellular Vesicles

Compositions useful for the methods of the present disclosure can be administered via, inter alia, localized injection, including catheter administration, systemic injection, localized injection, intravenous injection, intrauterine injection or parenteral administration. When administering a therapeutic composition described herein (e.g., a pharmaceutical composition), it will generally be formulated in a unit dosage injectable form (e.g. solution, suspension, or emulsion).

In any of the embodiments, there may be single or repeated administration of extracellular vesicles, including two, three, four, five or more administrations of extracellular vesicles. In some embodiments, the isolated EV is administered in 2 doses, 3 doses, 4 doses, 5 doses, 6 doses, 7, doses, 8 doses, 9 doses, 12 doses, 15 doses, 18 doses, or more. In some embodiments, the isolated EV is administered in 2 doses, 3 doses, 4 doses, 5 doses, 6 doses, 7, doses, 8 doses, 9 doses, 12 doses, 15 doses, 18 doses, or more within a week. In some embodiments, the extracellular vesicles may be administered continuously. Repeated or continuous administration may occur over a period of several hours (e.g., 1-2, 1-3, 1-6, 1-12, 1-18, or 1-24 hours), several days (e.g., 1-2, 1-3, 1-4, 1-5, 1-6 days, or 1-7 days) or several weeks (e.g., 1-2 weeks, 1-3 weeks, or 1-4 weeks) depending on the severity of the condition being treated. If administration is repeated but not continuous, the time in between administrations may be hours (e.g., 4 hours, 6 hours, or 12 hours), days (e.g., 1 day, 2 days, 3 days, 4 days, 5 days, or 6 days), or weeks (e.g., 1 week, 2 weeks, 3 weeks, or 4 weeks). In some embodiments, the isolated EV is administered at an interval of 12 hours, 24 hours, 48 hours, 72 hours, 4 days, 5, days, 6 days, or once per week. In some embodiments, the isolated EV is administered once daily for 2 days, for 3 days, for 4 days, for 5 days, or for a week The time between administrations may be the same or they may differ. As an example, if the symptoms of the disease appear to be worsening the extracellular vesicles may be administered more frequently, and then once the symptoms are stabilized or diminishing the extracellular vesicles may be administered less frequently. In some embodiments, the EVs can be administered upon onset of respiratory distress, such as ARDS, and can continue to be administered for at least the duration of the respiratory distress. In some embodiments, the EVs can be administered for most or all of the duration of mechanical ventilation. Such administration may reduce inflammation, either resulting from the underlying condition or the mechanical ventilation itself. Such administration may reduce deleterious effects of the mechanical ventilation. EVs can be administered to those having one or more risk factors for developing myocarditis, such as SAR-CoV-2 infection or infection with another virus.

EVs can be administered repeatedly in low dosage forms or as single administrations of high dosage forms. Low dosage forms may range from, without limitation, 1-50 micrograms per kilogram, while high dosage forms may range from, without limitation, 51-1000 micrograms per kilogram. It will be understood that, depending on the severity of the disease, the health of the subject, and the route of administration, inter alia, the single or repeated administration of low or high dose extracellular vesicles are contemplated.

The unit dose of EV may be phosopholipids of EVs per kg of subject being treated. In some embodiments, the effective dose of the isolated EV is 50 pmol of phosopholipids of EVs per kg of subject being treated (pmol/kg). In some embodiments, the effective dose of the isolated EV is from 20 to 500 pmol of phosopholipids of EVs per kg of subject being treated (pmol/kg). In some embodiments, the effective dose of the isolated EV is from 100 to 500 pmol of phosopholipids of EVs per kg of subject being treated (pmol/kg). In some embodiments, the effective dose of the isolated EV is from 200 to 500 pmol of phosopholipids of EVs per kg of subject being treated (pmol/kg). In some embodiment, the effective dose of the isolated EV is between 20-150 pmol/kg. In some embodiment, the effective dose of the isolated EV is between 25-100 pmol/kg. In some embodiment, the effective dose of the isolated EV is between 25-75 pmol/kg. In some embodiment, the effective dose of the isolated EV is between 40-60 pmol/kg. of phosopholipids of EVs per kg of subject being treated.

The EVs may be used in combination treatments. In some embodiments, the EVs are administered with a therapeutic agent comprising one or more of a phosphodiesterase type-5 (PDE5) inhibitor, a prostacyclin agonist, or an endothelin receptor antagonist. In some embodiments, wherein the isolated EV and the therapeutic agent are administered in the same composition. In some embodiments, the EVs and the therapeutic agent are administered in separate compositions, substantially simultaneously or sequentially. In some embodiments, the isolated EV and therapeutic agent are administered at an interval of 6 hours, 12, hours, 24 hours, 48 hours, 72 hours, 4 days, 5, days, 6 days, or once per week.

In some embodiments, the method further comprises administering a phosphodiesterase type-5 (PDE5) inhibitor as the therapeutic agent. In some embodiments, the PDE5 inhibitor comprises sildenafil, vardenafil, zapravist, udenafil, dasantafil, avanafil, mirodenafil, or lodenafil. In some embodiments, the PDE5 inhibitor is sildenafil. In some embodiments, the isolated EV and the phosphodiesterase type-5 (PDE5) inhibitor are administered in separate compositions, substantially simultaneously or sequentially. In some embodiments, the isolated EV and the phosphodiesterase type-5 (PDE5) inhibitor are administered in the same composition. In some embodiments, the isolated EV and the PDE5 inhibitor are administered in one or more doses. In some embodiments, the isolated EV and PDE5 inhibitor are administered at an interval of 6 hours, 12, hours, 24 hours, 48 hours, 72 hours, 4 days, 5, days, 6 days, or once per week. In some embodiments, the isolated EV is administered in 2 doses, 3 doses, 4 doses, 5 doses, 6 doses, 7 doses, 8 doses, 9 doses, 12 doses, 15 doses, 18 doses, or more, and wherein the PDE5 inhibitor is administered in 16 doses, 19 doses, 21 doses, 24 doses, 27 doses, 30 doses, 33 doses, 36 doses, 39 doses, 42 doses, 45 doses, 48 doses, 51 doses, 54 doses, 57 doses, 60 doses, 63 doses, 66 doses, or more. In some embodiments, the isolated EV is administered in 2 doses, 3 doses, 4 doses, 5 doses, 6 doses, 7 doses, 8 doses, 9 doses, 12 doses, 15 doses, 18 doses, or more within a week, and wherein the PDE5 inhibitor is administered in 16 doses, 19 doses, 21 doses, 24 doses, 27 doses, 30 doses, 33 doses, 36 doses, 39 doses, 42 doses, 45 doses, 48 doses, 51 doses, 54 doses, 57 doses, 60 doses, 63 doses, 66 doses, or more within a week.

In some embodiments, the EVs may be administered with a prostacyclin agonist. In some embodiments, the prostacyclin agonist comprises epoprostenol sodium, treprostinil, beraprost, ilprost, and a PGI2 receptor agonist. In some embodiments, the isolated EV and the prostacyclin agonist are administered at an interval of 6 hours, 12, hours, 24 hours, 48 hours, 72 hours, 4 days, 5, days, 6 days, or once per week. In some embodiments, the isolated EV and the prostacyclin agonist are administered in one or more doses. In some embodiments, the isolated EV and the prostacyclin agonist are administered in separate compositions, substantially simultaneously or sequentially. In some embodiments, the isolated EV and the prostacyclin agonist are administered in the same composition.

In some embodiments, the EV may be administered with an endothelin receptor agonist. In some embodiments, the isolated EV and the endothelin receptor agonist are administered in one or more doses. In some embodiments, the isolated EV and the endothelin receptor are administered at an interval of 6 hours, 12, hours, 24 hours, 48 hours, 72 hours, 4 days, 5, days, 6 days, or once per week. In some embodiments, the isolated EV and the endothelin receptor agonist are administered in separate compositions, substantially simultaneously or sequentially. In some embodiments, the isolated EV and the endothelin receptor agonist are administered in the same composition.

The extracellular vesicles may be used (e.g., administered) in pharmaceutically acceptable preparations (or pharmaceutically acceptable compositions), typically when combined with a pharmaceutically acceptable carrier. The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically-acceptable carrier” as used herein means a pharmaceutically-acceptable material, composition or vehicle, such as a liquid or solid filler, diluent, excipient, or solvent encapsulating material.

Such preparations may routinely contain pharmaceutically acceptable concentrations of salt, buffering agents, preservatives, compatible carriers, and may optionally comprise other (i.e., secondary) therapeutic agents. A pharmaceutically acceptable carrier is a pharmaceutically acceptable material, composition or vehicle, such as a liquid or solid filler, diluent, excipient, solvent or encapsulating material, involved in carrying or transporting a prophylactically or therapeutically active agent. Each carrier must be “acceptable” in the sense of being compatible with the other ingredients of the formulation and not injurious to the subject. Some examples of materials which can serve as pharmaceutically acceptable carriers include sugars, such as lactose, glucose and sucrose; salts such as sodium chloride; ethylenediaminetetraacetic acid (EDTA); glycols, such as propylene glycol; polyols, such as glycerin, sorbitol, mannitol and polyethylene glycol; esters, such as ethyl oleate and ethyl laurate; buffering agents, such as magnesium hydroxide and aluminum hydroxide; pyrogen-free water; isotonic saline; Ringer's solution; ethyl alcohol; phosphate buffer solutions; and other nontoxic compatible substances employed in pharmaceutical formulations.

The preparations are administered in effective amounts. An effective amount is that amount of an agent that alone stimulates the desired outcome. The absolute amount will depend upon a variety of factors, including the material selected for administration, whether the administration is in single or multiple doses, and individual patient parameters including age, physical condition, size, weight, and the stage of the disease. These factors are well known to those of ordinary skill in the art and can be addressed with no more than routine experimentation.

Other embodiments include a packaged and labelled pharmaceutical product. This article of manufacture or kit includes the appropriate unit dosage form in an appropriate vessel or container such as a glass vial or plastic ampoule or other container that is hermetically sealed. The unit dosage form should be suitable for pulmonary delivery for example by aerosol. Preferably, the article of manufacture or kit further comprises instructions on how to use including how to administer the pharmaceutical product. The instructions may further contain informational material that advises a medical practitioner, technician or subject on how to appropriately prevent or treat the disease or disorder in question. In other words, the article of manufacture includes instructions indicating or suggesting a dosing regimen for use including but not limited to actual doses, monitoring procedures, and other monitoring information.

As with any pharmaceutical product, the packaging material and container are designed to protect the stability of the product during storage and shipment. The kits may include MSC extracellular vesicles in sterile aqueous suspensions that may be used directly or may be diluted with normal saline for intravenous injection or use in a nebulizer, or dilution or combination with surfactant for intratracheal administration. The kits may therefore also contain the diluent solution or agent, such as saline or surfactant.

### EXAMPLES

The following examples are intended to provide those of ordinary skill in the art with a complete disclosure and description of how to make and use the methods and compositions described herein and are not intended to be limiting.

### Example 1—the EVs Reduce Inflammation by Inhibiting LPS-Induced TNF Alpha, and GRO

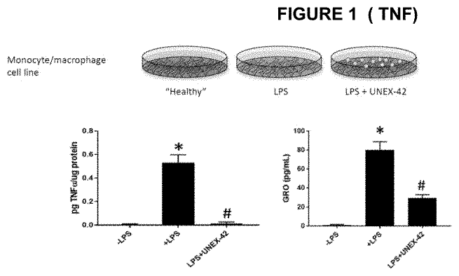

An in-vitro activation of THP-1 monocytes by bacterial lipopolysaccharide (LPS) induces the secretion of inflammatory cytokines, TNF alpha, and GRO. This example showed that UNEX-42 attenuated this response. See FIG. 1.

### Example 2—the EVs Enhance Expression of M2 (Anti-Inflammatory Macrophage Gene Expression in THP-1 Monocytes

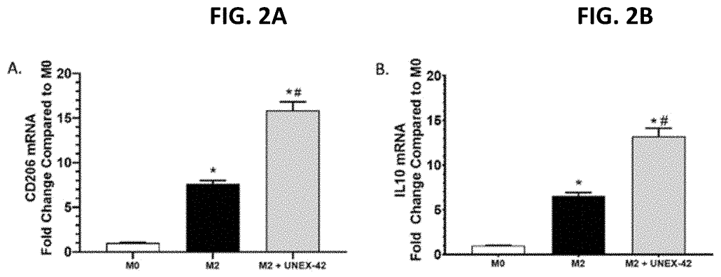

This Example showed that THP-1 cells polarized by a combination of IL-4 and IL-13 showed increase expression of CD206 and IL10. UNEX-42 increased expression of these genes by approximately 2-fold. See FIGS. 2A-2B.

### Example 3—the UNEX-42 EVs Decreased the Release of Neutrophil Chemoattractants, KCI and LIX in the Bronchoalveolar Lavage Fluid

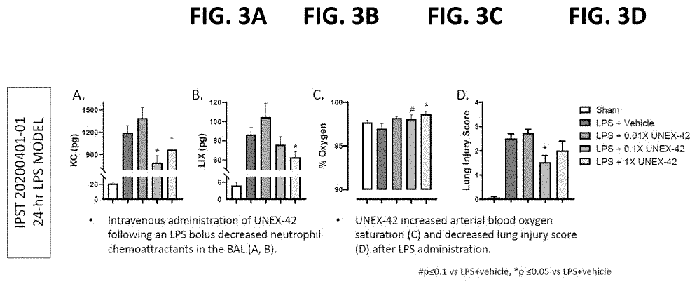

In a mouse model of acute lung injury induced by intratracheal administration of LPS, UNEX-42 decreased the release of neutrophil chemoattractants, KCI and LIX in the bronchoalveolar lavage (BAL) fluid. Mice treated with UNEX-42 further demonstrated improved lung function, as indicated by increased blood oxygen saturation, and improved histology as indicated by decreased lung injury score. It was also shown that UNEX-42 EVs inhibit LPS-induced expression of neutrophil chemoattractant KC and LIX. See FIGS. 3A-3D.

### Example 4—the UNEX-42 EVs Normalized the Secretion of Anti-Inflammatory Factors

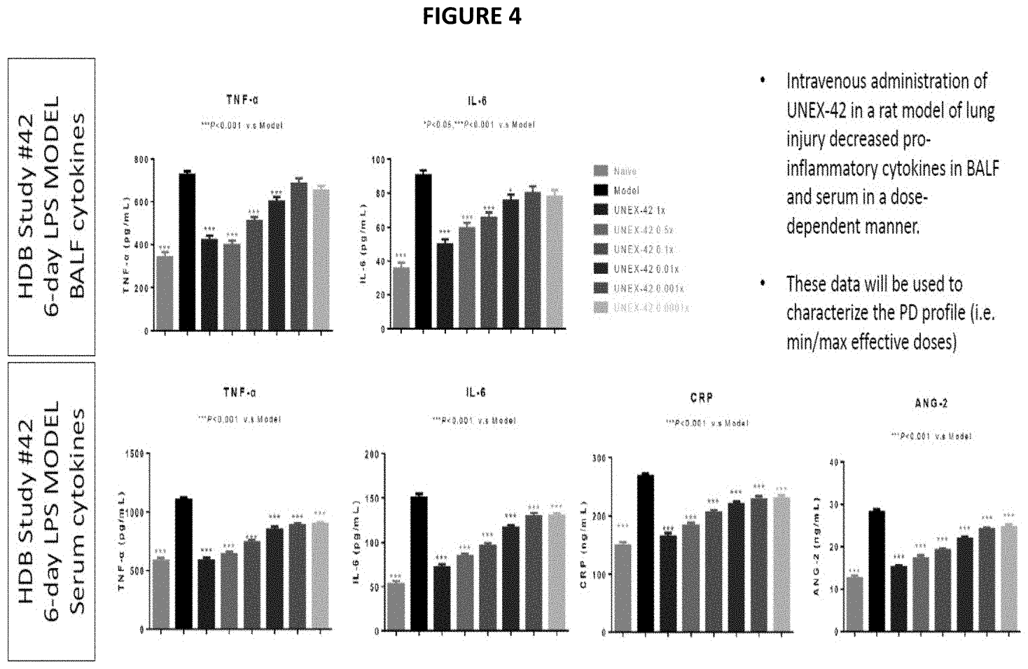

This example showed that UNEX-42 EVs increased expression of anti-inflammatory cytokine, IL-10, and decreased the secretion of inflammatory cytokines, IL-6 and TNF alpha in the bronchoalveolar lavage (BAL)—fluid in a rat model of acute lung injury induced by intratracheal administration of LPS. FIG. 4.

### Example 5—the EVs Prevent Cell Infiltration

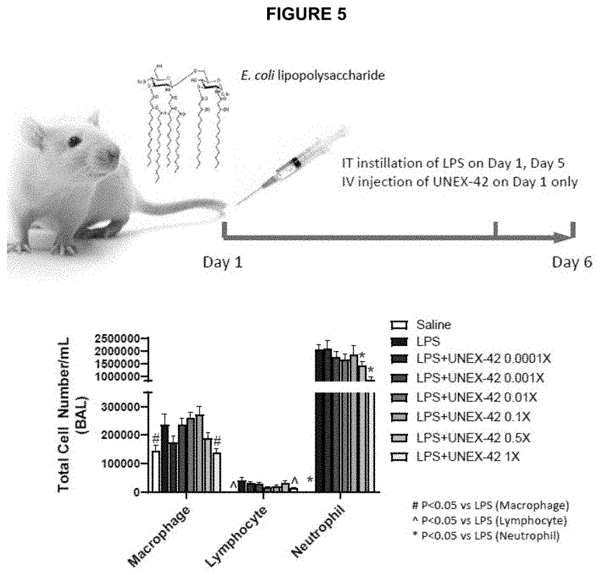

This example showed that UNEX-42 EVs reduced the number of cellular infiltrates in the lung, primarily neutrophils, in a rat model of acute lung injury induced by intratracheal administration of LPS. FIG. 5.

### Example 6—the EVs Reduced the Number of Cellular Infiltrates the Lung

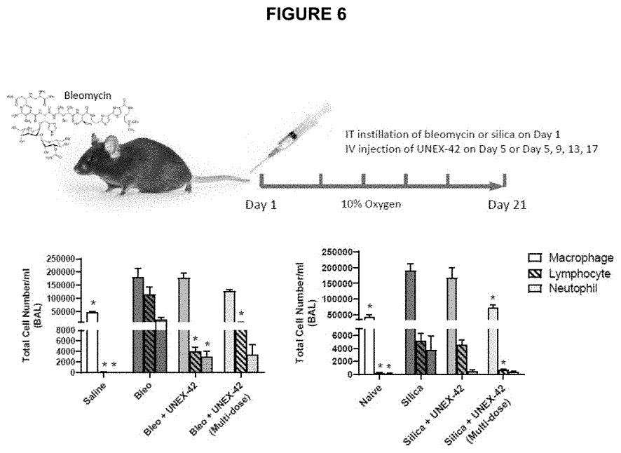

This example showed that UNEX-42 EVs reduced the number of cellular infiltrates the lung in mouse models of acute lung injury induced by intratracheal administration of bleomycin or silica. The reductions in cellular infiltrates were predominantly due to neutrophils, and with lesser reductions in lymphocytes and macrophages. FIG. 6.

### Example 7—the EVs can Increase the Presence of Cytotoxic T Cells

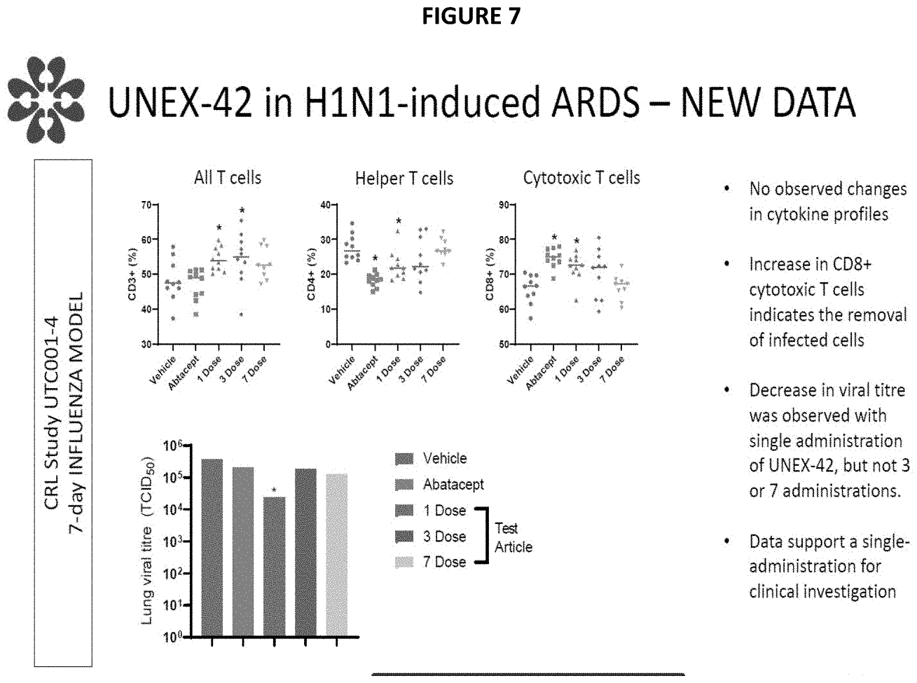

This example showed that UNEX-42 EVs increased the presence of CD8-positive cytotoxic T cells in a mouse model of H1N1 influenza-induced lung injury. See FIG. 7. Viral titers were reduced by a single administration of UNEX-42, but not with 3 or 7 administrations. See FIG. 7.

### Example 8—the EVs Promote Endothelial Cell Vessel Formation and Stabilization

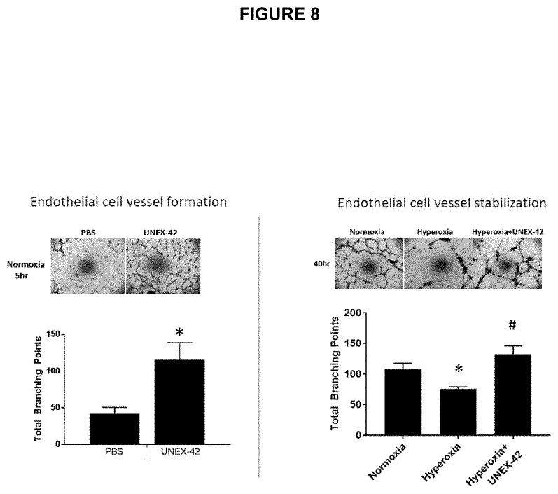

This example showed that UNEX-42 increased the complexity of the vascular networks as evidenced by the increase in the number of branchpoints. See FIG. 8. Furthermore, UNEX-42 prevented the loss of vascular networks caused by short-term exposure to high oxygen levels. FIG. 8.

Cells were treated with PBS or UNEX-42 and then exposed to normoxia (21% O2) or hyperoxia (97% O2) for 40 hours to model hyperoxia-mediated vascular network damage. Tube branching points were evaluated, and exposure of control cells to hyperoxia resulted in a deterioration of the vascular network, as indicated by a reduction in branching points, whereas UNEX-42 pre-treatment fully prevented this deterioration as shown in FIG. 8.

### Example 9—Improved Metabolic Function by EVs Derived from MSC

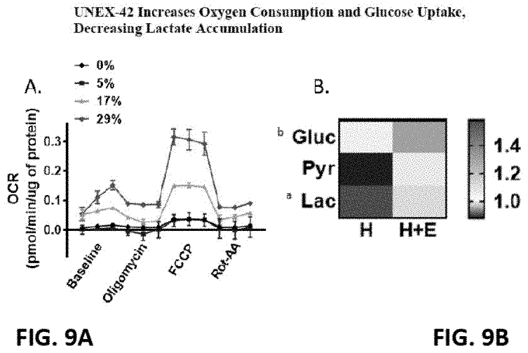

In pulmonary artery smooth muscle cells exposed to an hypoxic environment, UNEX-42 increased oxygen consumption rate (OCR), increased glucose uptake, and decreased lactate accumulation. See FIGS. 9A-9B.

### Example 10—Cellular Salvage by EVs Derived from MSC

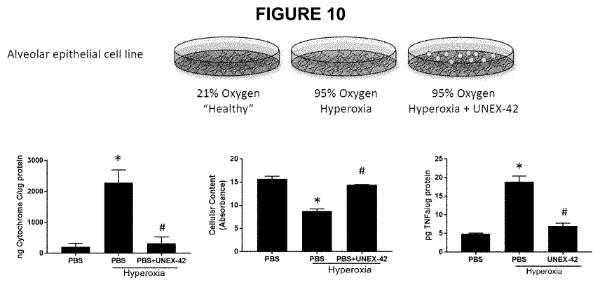

In the alveolar epithelial cell line, A549, hyperoxia induced apoptosis as evidenced by the release of cytochrome c, and concomitant loss of cellular content (i.e. DNA). Further, hyperoxia induced the release of TNF alpha. UNEX-42 EVs prevented this hyperoxia-induced damage as shown in FIG. 10.

All patents, patent applications, publications and references cited herein are incorporated by reference in their entirety to the extent as if they were individually incorporated by reference.

What is claimed is:

1. A method of treating, preventing, or reducing the severity of myocarditis, comprising administering to a subject in need thereof an effective dose of an isolated extracellular vesicle (EV).

2. The method of claim 1, wherein the myocarditis is caused by an infection, a toxic drug, or an autoimmune disease or disorder.

3. The method of claim 2, wherein the infection is a bacterial infection, a fungal, or a viral infection.

4. The method of claim 3, wherein the viral infection is caused by Coxsackievirus B virus, or SARS-CoV-2.

5. The method of claim 1, wherein the method treats myocarditis caused by SARS-CoV-2 infection.

6. The method of claim 1, wherein the method treats, prevents or reduces the severity of the myocarditis by reducing inflammation in the heart.

7. The method of claim 6, wherein the EVs reduce inflammation in the heart by reducing a level of a pro-inflammatory factor.

8. The method of claim 7, wherein the pro-inflammatory factor comprises TNF-α, IL-1, IL-6, IL-8, TGFβ, IFNγ, C-reactive protein (CRP), RAGE, or combinations thereof.

9. The method of claim 8, wherein the EVs reduce inflammation in the heart by increasing a level of an anti-inflammatory factor.

10. The method of claim 9, wherein the anti-inflammatory factor comprises IL-10.

11. The method of claim 1, wherein the method reduces infiltration of an immune cell to the heart.

12. The method of claim 11, wherein the immune cell comprises neutrophils, dendritic cells, monocytes, macrophages, or lymphocytes.

13. The method of claim 1, wherein the method reduces vascular damage in the heart of the subject.

14. The method of claim 13, wherein the vascular damage is reduced by endothelial cell vessel formation or stabilization.

15. The method of claim 13, wherein the vascular damage is caused by a viral infection.

16. The method of claim 15, wherein the viral infection is caused by Coxsackievirus B virus, or SARS-CoV-2.

17. The method of claim 1, wherein the method improves cellular metabolism in the heart of the subject.

18. The method of claim 16, wherein improved cellular metabolism is measured by oxygen consumption, glucose uptake, or lactate accumulation.

19. The method of claim 1, wherein the method prevents apoptosis.

20. The method of claim 19, wherein prevention of apoptosis is determined by measuring cytochrome c release.

21. The method of claim 1, wherein the isolated EV contains one more proteins selected from the group consisting of KRT19, TUBB, TUBB2A, TUBB2B, TUBB2C, TUBB3, TUBB4B, TUBB6, CFL1 (HEL-S-15), VIM, EEF1A1, EEF1A1P5, PTI-1, EEF1A1L14, EEFA2, ENPP1, NTSE, HSPA8 (HEL-S-72p), RAB10, CD44, MMP2, CD109, and DKFZp686P132.

22. The method of claim 21, wherein the EV contains one or more proteins selected from the group consisting of CD44, CD109, NTSE, MMP2 and HSPA8.

23. The method of claim 1, wherein the subject is human.

24. The method of claim 1, wherein the administration occurs following a diagnosis of myocarditis in the subject.

25. The method of claim 1, wherein the administration occurs during a viral infection.

26. An isolated EV comprising one or more proteins selected from the group consisting of KRT19, TUBB, TUBB2A, TUBB2B, TUBB2C, TUBB3, TUBB4B, TUBB6, CFL1 (HEL-S-15), VIM, EEF1A1, EEF1A1P5, PTI-1, EEF1A1L14, EEFA2, ENPP1, NT5E, HSPA8 (HEL-S-72p), RAB10, CD44, MMP2, CD109, and DKFZp686P132.
